(19) United States
(12) Patent Application Publication
(10) Pub. No.: US 2001/0055594 A1
(43) Pub. Date: Dec. 27, 2001

(54) Use of certain drugs for treating nerve root injury

(76) Inventors: Kjell Olmarker, Molndal (SE); Bjorn Rydevik, Goteborg (SE)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 09/826,893

(22) Filed: Apr. 6, 2001

Related U.S. Application Data
(63) Continuation-in-part of application No. 09/743,852, filed on Jan. 17, 2001.
Application No. 09/743,852 is a 371 of international application No. PCT/SE99/01671, filed on Sep. 23, 1999.

(30) Foreign Application Priority Data
Sep. 25, 1998 (SE) 9803276-6
Oct. 29, 1998 (SE) 9803710-4

(51) Int. Cl. A61K 39/395; A61K 38/00

(52) U.S. Cl. 424/145.1; 514/2

(57) ABSTRACT

The present invention relates to pharmaceutical compositions for the treatment of spinal disorders caused by the liberation of TNF-α comprising an effective amount of a TNF-α inhibitor, as well as a method for treatment of such disorders, and the use of TNF-α inhibitors in the preparation of pharmaceutical compositions for such treatment.

This application is a continuation-in-part application of the U.S. patent application entitled “Use of Certain Drugs for Treating Nerve Root Injury” filed Jan. 17, 2001 as a National Stage of International Application No. PCT/SE99/01671, filed Sep. 23, 1999 that designates the United States of America and was published under PCT Article 21(2) in English on Apr. 6, 2000 and claims benefit of Swedish Applications 9803276-6 and 9803710-4 filed respectively on Sep. 25, 1998 and Oct. 29, 1998. These applications are herein incorporated by reference in their entirety.

### TECHNICAL FIELD

The present invention relates to a method for treating nerve disorders in a mammal or a vertebrate by administering a TNF-α inhibitor. The invention also relates to the use of a TNF-α inhibitor in the preparation of pharmaceutical compositions for the treatment of nerve root injury.

The object of the present invention is to obtain a possibility to treat nerve root injury induced by disk herniation, which may turn up as a radiating pain in the arm or leg (sciatica), by blocking disk related cytokines.

### BACKGROUND OF THE INVENTION

Disk herniation is a troublesome disorder, which can cause pronounced pain and muscle dysfunction, and thereby loss of ability to work. A herniation may occur in any disk in the spine but herniations in the lumbar and the cervical spine are most common. A disk herniation in the cervical spine may induce radiating pain and muscle dysfunction in the arm. Herniation in the lumbar spine may induce radiating pain and muscle dysfunction in the leg. The radiating pain in the leg is generally referred to a “sciatica”. Disk herniation will cause trouble to a varying degree, and the pain may last for one or two months or in severe cases up to 6 months. The arm or leg pain that can occur as a result of disk herniation can be very intense and may thus affect the individual patient's whole life situation during the sickness period.

U.S. Pat. No. 5,703,092 discloses the use of hydroxamic acid compounds and carbocyclic acids as metalloproteinase and TNF inhibitors, for the treatment of arthritis and other related inflammatory diseases. No use of these compounds for the treatment of nerve root injuries is disclosed or suggested.

U.S. Pat. No. 4,925,833 discloses the use of tetracyclines to enhance bone protein synthesis and treatment of osteoporosis.

U.S. Pat. No. 4,666,897 discloses inhibition of mammalian collagenolytic enzymes by administering tetracyclines. The collagenolytic activity is manifested by excessive bone resorption, periodontal disease, rheumatoid arthritis, ulceration of cornea, or resorption of skin or other connective tissue collagen.

Neither of these latter two documents mentions nerve root injury or the treatment thereof.

### SUMMARY OF THE INVENTION

It is an object of the invention to provide novel and improved methods for inhibiting the action of TNF-α for treating disorders in a subject by administering a TNF-α inhibitor comprising the step of administering to said subject a therapeutically effective dosage of said TNF-α inhibitor, wherein said TNF-α inhibitor is a monoclonal antibody selected from CDP-571 (HUMICADE™) D2E7, and CDP-870.

Alternatively the TNF-α inhibitor used in the above method can be lactoferrin, CT3, ITF-2357, PD-168787, CLX-1100, M-PGA, NCS-700, PMS-601, RDP-58, TNF-484A, PCM-4, CBP-1011, SR-31747, AGT-1, Solimastat, CH-3697, NR58-3.14.3, RIP-3, Sch-23863 and SH-636.

The subject which can be treated by these methods include any vertebrate, preferably mammals, and of those, most preferably humans.

It is a more specific object of the invention to provide a novel pharmaceutical composition for treating nerve disorders in a subject comprising a therapeutically effective amount of a TNF-α inhibitor is a monoclonal antibody selected from the group consisting of CDP-571 (HUMICADE™) D2E7, and CDP-870, and a pharmaceutically acceptable carrier, wherein said pharmaceutical composition inhibits nerve injury when administered to said subject. The pharmaceutical composition alternatively can comprise one or more of these agents, or can comprise, alone or in combination, any of the agents discussed herein.

In another embodiment, the methods and pharmaceutical compositions described herein can be used to treat such nerve disorders as spinal disorders, nerve root injuries, a nerve disorder caused by or associated with a herniated disc(s), a nerve disorder involving pain, a nucleus pulposus-induced nerve injury, a spinal cord compression and sciatica.

With the foregoing and other objects, advantages and features of the invention that will become hereinafter apparent, the nature of the invention may be more clearly understood by reference to the following detailed description of the preferred embodiments of the invention and to the appended claims.

### DESCRIPTION OF THE PRESENT INVENTION

It has now surprisingly been shown possible to be able to treat nerve root injuries, or at least alleviate the symptoms of nerve root injuries by using a pharmaceutical composition comprising an therapeutically active amount of a TNF-α inhibitor. TNF-α inhibitors, include but are not limited to, metalloproteinase (MMP) inhibitors (excluding methylprednisolone), tetracyclines, chemically modified tetracyclines, quinolones, corticosteroids, thalidomide, lazaroides, pentoxyphylline, hydroxamic acid derivatives, napthopyrans, soluble cytokine receptors, monoclonal antibodies towards TNF-α, amrinone, pimobendan, vesnarinone, phosphodiesterase III inhibitors, lactoferrin and lactoferrin derived analogous, and melatonin in the form of bases or addition salts together with a pharmaceutically acceptable carrier.

By “therapeutically active amount” is intended an amount that will lead to the desired therapeutical effect, i.e., an amount that will lead to an improvement of the patient's condition.

By “mammal” is meant to include but is not limited to primate, human, canine, porcine, equine, murine, feline, caprine, ovine, bovine, lupine, camelid, cervidae, rodent, avian and ichthyes. By animal is meant to include any vertebrate animal wherein there is a potential for nerve root injury.

As used herein, the term “antibody” is meant to refer to complete, intact antibodies, and Fab fragments, scFV, and F(ab)₂ fragments thereof. Complete, intact antibodies include monoclonal antibodies such as murine monoclonal antibodies (mAb), chimeric antibodies, humanized antibodies and human. The production of antibodies and the protein structures of complete, intact antibodies, Fab fragments, scFv fragments and F(ab)₂ fragments and the organization of the genetic sequences that encode such molecules, are well known and are described, for example, in Harlow et al., ANTIBODIES: A LABORATORY MANUAL, Cold Spring Harbor Laboratory, Cold Spring Harbor, N.Y. (1988) and Harlow et al., USING ANTIBODIES: A LABORATORY MANUAL, Cold Spring Harbor Press, 1999, which are herein incorporated by reference in their entirety.

By “epitope” is meant a region on an antigen molecule to which an antibody or an immunogenic fragment thereof binds specifically. The epitope can be a three dimensional epitope formed from residues on different regions of a protein antigen molecule, which, in a naive state, are closely apposed due to protein folding. “Epitope” as used herein can also mean an epitope created by a peptide or hapten portion of TNF-α and not a three dimensional epitope. Preferred epitopes are those wherein when bound to an immunogen (antibody, antibody fragment, or immonogenic fusion protein) results in inhibited or blocked TNF-α activity.

By “TNF-α blocking” is meant a compound or composition which blocks, inhibits or prevents the activity of TNF-α.

Compounds that possess TNF-α inhibitory activity are for example tetracyclines, (e.g., tetracycline, doxycycline, lymecycline, oxytetracycline, minocycline), and chemically modified tetracyclines (e.g., dedimethylamino-tetracycline), hydroxamic acid compounds, carbocyclic acids and derivatives, thalidomide, lazaroides, pentoxyphylline, napthopyrans, soluble cytokine receptors, monoclonal antibodies towards TNF-α, amrinone, pimobendan, vesnarinone, phosphodiesterase III inhibitors, lactoferrin and lactoferrin derived analogs, melatonin, norfloxacine, ofloxacine, ciprofloxacine, gatifloxacine, pefloxacine, lomefloxacine, and temafloxacine. These compounds can be present as bases or in the form of addition salts, whichever possesses the best or preferred pharmaceutical effect, and best property to be brought into a suitable pharmaceutical composition. A more complete list is given below.

As stated above, there are several different types of TNF-α blocking substances and pharmacological preparations that may be used according to the invention, and those substances may be grouped in different subclasses:

SPECIFIC TNF-A BLOCKING SUBTANCES

| | |
|---|---|
| Monoclonal Antibodies | infliximab, CDP-571, |
| | (HUMICADE ™), D2E7, CDP-870 |
| Soluble Cytokine Receptors | etanercept, lenercept, pegylated TNF- |
| | receptor type I, TBP-1 |
| TNF-receptor antagonists | |
| Antisense oligonucleotides | ISIS-104838 |

NON-SPECIFIC TNF-A BLOCKING SUBSTANCES

| | |
|---|---|
| MMP-inhibitors | (or TACE-inhibitors, i.e. TNF Alpha |
| | Converting Enzyme-inhibitors), |
| | AG3340 (Prinomastat), Batimastat and |
| | Marimastat |
| Tetracyclines | Doxycycline, Lymecycline, |
| | Oxitetracycline, Tetracycline, |
| | Minocycline and synthetic tetracycline |
| | derivatives, such as CMT (i.e., |
| | Chemically Modified Tetracyclines |
| | such as KB-R7785; TIMP1 and 2, |
| | adTIMP2) |
| Quinolones | Norfloxacin, Levofloxacin, Enoxacin, |
| | Sparfloxacin, Temafloxacin, |
| | Moxifloxacin, Gatifloxacin, |
| | Gemifloxacin, Grepafloxacin, |
| | Trovafloxacin, Ofloxacin, |
| | Ciprofloxacin, Pefloxacin, |
| | Lomefloxacin and Temafloxacin |
| Thalidomide derivatives | Selective Cytokine inhibitors (SelCID), |
| | such as thalidomide derivatives such as |
| | CC-1088, CDC-501, and CDC-801 |
| | (ROQUININEX ®) |
| Lazaroids | non-glucocorticoid 21-aminosteroids |
| | such as U-74389G (16-desmethyl |
| | tirilazad) and U-74500 |
| Prostaglandins | Iloprost (prostacycline) |
| Cyclosporins | |
| Pentoxifyllin derivatives | |
| Hydroxamic acid derivatives | |
| Napthopyrans | |
| Phosphodiesterase I, II, III, | CC-1088, Ro 20-1724, rolipram, |
| IV, and V-inhibitors | amrinone, pimobendan, vesnarinone, |
| | SB 207499 (ARIFLO ®) |
| Melancortine agonists | HP-228 |
| Other TNF-α blocking agents | Lactoferrin; CT3; ITF-2357; PD- |
| | 168787; CLX-1100; M-PGA; NCS- |
| | 700; PMS-601; RDP-58; TNF-484A; |
| | PCM-4; CBP-1011; SR-31747; AGT- |
| | 1; Solimastat; CH-3697; NR58-3.14.3; |
| | RIP-3; Sch-23863; Yissum project no. |
| | 11649; Pharma projects no. 6181, 6019 |
| | and 4657; SH-636 |

Also contemplated are the pharmaceutically acceptable bases and salts of the substances listed above.

Preferred groups of TNF-α blocking substances for use according to the present invention are soluble cytokine receptors, monoclonal antibodies, and tetracyclines or chemically modified tetracyclines.

Two preferred substances for use according to the present invention are the monoclonal antibodies, D2E7 and CDP-870.

D2E7 is a fully humanized monoclonal antibody directed against human TNF-α, which has been developed by Knoll and Cambridge Antibody Technology. A transgenic recombinant version of this antibody is under development by Genzyme Transgenic. The invention contemplates any antibody that binds to the same epitope as D2E7 or that has the same TNF-α inhibitory effect as D2E7. Preferably the antibody is primatized, humanized or human.

CDP-870 (or CDP 870) is a humanized antibody fragment with high affinity to TNF-α. It has been developed by Celltech Group plc, and is marketed by Pharmacia Corporation. The invention contemplates any antibody, antibody fragment or immunogen that binds to the same epitope as CDP-870 or that has the same TNF-α inhibitory activity as CDP-870. Preferably the antibody, antibody fragment or immunogen has the same or similar TNF-α inhibitory activity. Preferably the antibody, antibody fragment or immunogen is primatized, humanized or human.

Further, the active component may be a substance inhibiting a compound triggered by the release of TNF-α or part of a TNF-α cascade that is associated with nerve root injury, such as interferon-gamma (INFγ), interleukin-1 (IL-1), and nitrogen oxide (NO) in the form of base or addition salts.

It is possible to use either one or two or more substances according to the invention in the treatment, for example, of low back pain (LBP). When two or more substances are used they may be administered either simultaneously or separately.

The substances according to the invention may also be administered in combination with other drugs or compounds, provided that these other drugs or compounds do not eliminate the desired effects according to the present invention, i.e., the effect on TNF-α.

The invention further relates to a method for inhibiting the symptoms of nerve root injury.

The effects of doxycycline, soluble cytokine-receptors, and monoclonal cytokine-antibodies have been studied and representative methods used and results obtained are disclosed below. Although the present invention has been described in detail with reference to examples herein, it is understood that various modifications can be made without departing from the spirit of the invention, and would be readily known to the skilled artisan.

The compounds of the invention can be administered in a variety of dosage forms, e.g., orally (per os), in the form of tablets, capsules, sugar or film coated tablets, liquid solutions; rectally, in the form of suppositories; parenterally, e.g., intramuscularly (i.m.), subcutaneous (s.c.), intracerebroventricular (i.c.v.), intrathecal (i.t.), epidurally, transepidermally or by intravenous (i.v.) injection or infusion; by inhalation; or intranasally.

The therapeutic regimen for the different clinical syndromes may be adapted to the disease or condition, medical history of the subject as would be know to the skilled artisan or clinician. Factors to be considered, but are not limited to the route of administration, the form in which the compound is administered, the age, weight, sex, and condition of the subject involved.

For example, the oral route is employed, in general, for all conditions, requiring such compounds. In emergency cases, preference is sometimes given to intravenous injection. For these purposes, the compounds of the invention can be administered, for example, orally at doses ranging from about 20 to about 1500 mg/day. Of course, these dosage regimens may be adjusted to provide the optimal therapeutic response depending on the subject's condition.

The nature of the pharmaceutical composition containing the compounds of the invention in association with pharmaceutically acceptable carriers or diluents will, of course, depend upon the desired route of administration. The composition may be formulated in the conventional manner with the usual ingredients. For example, the compounds of the invention may be administered in the form of aqueous or oily solutions or suspensions, tablets, pills, gelatin capsules (hard or soft ones), syrups, drops or suppositories.

For oral administration, the pharmaceutical compositions containing the compounds of the invention are preferably tablets, pills or gelatine capsules, which contain the active substance or substances together with diluents, such as lactose, dextrose, sucrose, mannitol, sorbitol, cellulose; lubricants, e.g., silica, talc, stearic acid, magnesium or calcium stearate, and/or polyethylene glycols; or they may also contain binders, such as starches, gelatine, methyl cellulose, carboxymethylcellulose, gum arabic, tragacanth, polyvinylpyrrolidone; disaggregating agents such as starches, alginic acid, alginates, sodium starch glycolate, microcrystalline cellulose; effervescing agents, such a carbonates and acids; dyestuffs; sweeteners; wetting agents, such as lecithin, polysorbates, laurylsulphates; and in general non-toxic and pharmaceutically inert substances used in the formulation of pharmaceutical compositions. Said pharmaceutical compositions may be manufactured in known manners, e.g., by means of mixing, granulating, tableting, sugar-coating or film-coating processes. Film providing compounds can be selected to provide release in the right place or at the appropriate time in the intestinal tract with regard to absorption and maximum effect. Thus pH-dependent film formers can be used to allow absorption in the intestines as such, whereby different phthalates are normally used or acrylic acid/methacrylic acid derivatives and polymers.

The liquid dispersions for oral administration may be, e.g., syrups, emulsions, and suspensions.

The syrups may contain as carrier, e.g., saccharose, or saccharose with glycerine and/or mannitol and/or sorbitol.

Suspensions and emulsions may contain as Garner, e.g., a natural gum, such as gum arabic, xanthan gum, agar, sodium alginate, pectin, methyl cellulose, carboxymethylcellulose, polyvinyl alcohol.

The suspension or solutions for intramuscular injections may contain together with the active compound, a pharmaceutically acceptable carrier, such as e.g., sterile water, olive oil (or other vegetable or nut derived oil), ethyl oleate, glycols”, e.g., propylene glycol, and if so desired, a suitable amount of lidocaine hydrochloride. Adjuvants for triggering the injection effect can be added as well.

The solutions for intravenous injection or infulsion may contain as carrier, e.g., sterile water, or preferably, a sterile isotonic saline solution, as well as adjuvants used in the field of injection of active compounds. Such solutions would also be suitable for i.m. and i.c.v. injection.

The suppositories may contain together with the active compounds, a pharmaceutically acceptable carrier, e.g., cocoa-butter polyethylene glycol, a polyethylene sorbitan fatty acid ester surfactant or lecithin.

Examples of suitable doses of the active agents contemplated for different administration routes are given below.

| | | | |
|---|---|---|---|
| Per os | 10-300 | mg | |
| i.m. | 25-100 | mg | |
| i.v. | 2.5-25 | mg | |
| i.t. | 0.1-25 | mg | (daily - every 3^{rd} month) |
| inhalation | 0.2-40 | mg | |
| transepidermally | 10-100 | mg | |
| intranasally | 0.1-10 | mg | |
| s.c. | 5-10 | mg | |
| i.c.v. | 0.1-25 | mg | (daily - every 3^{rd} month) |
| epidurally | 1-100 | mg | |

These ranges are approximate (e.g., about 1 to about 100) and may vary depending on the specific agent being administered and the nature of the disorder in the subject.

Examples of suitable doses for different TNF-α inhibitors are given in the table below. Dosages for all the compounds discussed herein will vary depending on the route of administration, the condition and the medical history of the patient.

| TNF-α | | More | Most |
|---|---|---|---|
| blocking substance | Preferred | preferred | preferred |
| and administration route | dosage | dosage | dosage |
| Lenercept | | | |
| i.v. | 5-200 | 10-100 | 30-80 |
| | (all given in mg for administration | | |
| | once every 4th week) | | |
| TBP-1 | | | |
| i.v. | 5-200 | 10-100 | 30-80 |
| | (all given in mg for administration | | |
| | once every 4th week) | | |
| CDP-571 | | | |
| (HUMICADE ®) | | | |
| i.v. | 1-100 | 5-10 | 5-10 |
| | (all given in mg/kg body weight for | | |
| | administration as a single dose) | | |
| D2E7 | | | |
| i.v. | 0.1-50 | 0.5-10 | 1-10 |
| s.c. | 0.1-50 | 0.5-10 | 1-10 |
| | (all given in mg/kg body weight for | | |
| | administration as a single dose) | | |
| Iloprost | | | |
| i.v | 0.1-2000 | 1-1500 | 100-1000 |
| | (all given in μg/kg body weight/day) | | |
| intranasally | 50-250 | 100-150 | 100-150 |
| | (all given in μg/day) | | |
| Thalidomide | 100-1200 | 300-1000 | 500-800 |
| | (all given in μg/day) | | |
| CC-1088 | | | |
| Per os | 50-1200 | 200-800 | 400-600 |
| | (all given in mg/day) | | |
| CDP-870 | | | |
| i.v. | 1-50 | 2-10 | 3-8 |
| | (all given in mg/kg body weight for | | |
| | administration once every 4th week) | | |
| HP-228 | | | |
| i.v. | 5-100 | 10-50 | 20-40 |
| | (all given in μg/kg body weight) | | |
| ISIS-10483 | | | |
| Per os | 1-100 | 10-50 | 20-50 |
| S.c. | 1-100 | 10-50 | 20-50 |
| i.v. | 1-100 | 10-50 | 20-50 |
| | (all given in mg) | | |
| ARIFLO ® | | | |
| (SB 207499 | | | |
| Per os | 10-100 | 30-60 | 30-45 |
| | (all given in mg/day) | | |
| KB-R7785 | | | |
| s.c. | 100-500 | 100-300 | 150-250 |
| | (all given in mg/kg body weight/day) | | |
| CDC-501 | | | |
| Per os | 50-1200 | 200-800 | 400-600 |
| | (all given in mg/day) | | |
| CDC-801 | | | |
| (ROQUININEX ®) | | | |
| Per os | 50-1200 | 200-800 | 400-600 |
| | (all given in mg/day) | | |
| Prinomastat, | | | |
| Batimastat, and | | | |
| Marimastat | | | |
| Per os | 1-250 | 5-100 | 10-50 |
| | mg | mg | mg |
| | (all given in mg twice/day) | | |
| Linomide | | | |
| Per os | 0.1-25 | 5-20 | 10-15 |

(all given in mg/kg body weight/day)

### Incorporation by Reference and Examples

Although the present invention has been described in detail with reference to examples below, it is understood that various modifications can be made without departing from the spirit of the invention, and would be readily known to the skilled artisan. All publications cited herein are herein incorporated by reference in their entirety.

### EXAMPLES

### Example 1

### Study Design

The effects of nucleus pulposus and various treatments to block TNF-α activity were evaluated in an experimental set-up using immunohistochemistry and nerve conduction velocity recordings.

### Summary of Background Data

A meta-analysis of observed effects induced by nucleus pulposus reveals that these effects might relate to one specific cytokine, Tumor Necrosis Factor alpha, (TNF-α).

### Objectives

To assess the presence of TNF-α in pig nucleus pulposus cells and to see if blockage of TNF-α also blocks the nucleus pulposus-induced reduction of nerve root conduction velocity.

### Methods

Series-1: Cultured nucleus pulposus-cells were immunohistologically stained with a monoclonal antibody for TNF-α.

Series-2: Nucleus pulposus was harvested from lumbar discs and applied to the sacrococcygeal cauda equina in 13 pigs autologously. Four pigs received 100 mg of doxycycline intravenously, 8 pigs had a blocking monoclonal antibody to TNF-α applied locally in the nucleus pulposus, and 4 pigs remained non-treated (controls). Three days after the application the nerve root conduction velocity was determined over the application zone by local electrical stimulation.

Series-3: Thirteen pigs had autologous nucleus pulposus placed onto their sacrococcygeal cauda equina similar to series-2. Five pigs (bodyweight 25 kg) received REMICADE™ (infliximab) 100 mg i.v. preoperatively, and 8 pigs received ENBREL™ (etanercept) 12.5 mg s.c. preoperatively and additionally 12.5 mg s.c. three days after the operation. Seven days after the nucleus pulposus-application the nerve root conduction velocity was determined over the application zone by local electrical stimulation according to series-2.

### Results

Series-1: TNF-α was found to be present in the nucleus pulposus-cells.

Series-2: The selective antibody to TNF-α limited the reduction of nerve conduction velocity, although not statistically significant as compared to the control series. However, treatment with doxycycline significantly blocked the nucleus pulposus-induced reduction of conduction velocity.

Series-3: Both drugs (infliximab, and etanercept) blocked the nucleus pulposus induced nerve injury efficiently. Normal average nerve conduction velocities were found after 15 treatment with both of these two drugs.

### Conclusion

For the first time a specific substance, Tumor Necrosis Factor-alpha, has been linked to the nucleus pulposus-induced effects of nerve roots after local application. Although the effects of this substance may be synergistic with other similar substances, the data of the present study may be of significant importance for the continued understanding of nucleus pulposus' biologic activity, and might also be of potential use for future treatment strategies of sciatica and other nerve root injury conditions or related conditions.

After previously being considered as just a biologically inactive tissue component compressing the spinal nerve root at disc herniation, the nucleus pulposus has recently been found to be highly active, inducing both structural and functional changes in adjacent nerve roots when applied epidurally (24, 37, 38, 41, 42). It has thereby been established that autologous nucleus pulposus may induce axonal changes and a characteristic myelin injury (24, 38, 41, 42), increased vascular permeability (9), infra vascular coagulation (24, 36), and that membrane-bound structure or substances of the nucleus pulposus-cells are responsible for these effects (24, 37). The effects have also been found to be efficiently blocked by methylprednisolone and cyclosporin A (2, 38). When critically looking at these data, one realizes that there is at least one cytokine that relates to all of these effects, TNF-α.

To assess if TNF-α may be involved in the nucleus pulposus induced nerve root injury, the presence of TNF-α in nucleus pulposus-cells was assessed and was studied if the nucleus pulposus-induced effects could be blocked by doxycycline, a soluble TNF-receptor, and a selective monoclonal TNF-α antibody, the latter administered both locally in the nucleus pulposus and systemically.

### Example 2

### Material and Methods

Series-1, Presence of TNF-α in pig nucleus pulposus-cells:

Nucleus pulposus (NP) from a total of 13 lumbar and thoracic discs were obtained from 10 pigs, which were used for other purposes. NP was washed once in Ham's F12 medium (Gibco BRL, Paisley, Scotland) and then centrifuged and suspended in 5 ml of collagenase solution in Ham's F12 medium (0.8 mg/ml, Sigma Chemical Co., St Louis, Mo., USA) for 40 minutes, at 37° C. in 25 cm² tissue culture flasks. The separated NP-cell pellets were suspended in DMEM/F12 1:1 medium (Gibco BRL, Paisley, Scotland) supplemented with 1% L-glutamine 200 mM (Gibco BRL, Paisley, Scotland), 50 mg/ml gentamycine sulphate (Gibco BRL, Paisley, Scotland) and 10% fetal calf serum (FCS), (Gibco BRL, Paisley, Scotland). The cells were cultured at 37° C. and 5% CO₂ in air for 3-4 weeks and then cultured directly on tissue culture treated glass slides (Becton Dickinson & Co Labware, Franklin Lakes, N.J., USA). After 5 days on the glass slides, the cells were fixed in situ by exposing the slides to acetone for 10 minutes. After blocking irrelevant antigens by application of 3% H₂O₂ (Sigma Chemical Co., St Louis, Mo., USA) for 30 minutes and Horse Serum (ImmunoPure ABC, peroxidase mouse IgG staining kit nr.32028, Pierce, Rockford, Ill.) for 20 minutes, the primary antibody (Anti-pig TNF-α monoclonal purified antibody, Endogen, Cambridge, Mass., USA) was applied over night at +40° C., diluted at 1:10, 1:20 and 1:40 dilutions. For control, BSA (bovine serum albumin, Intergen Co, New York, USA) suspended in PBS (phosphate buffered saline, Merck, Darmstadt, Germany) was applied in the same fashion. The next day the cells were washed with 1% BSA in PBS and the secondary antibody (ImmunoPure ABC, peroxidase mouse IgG staining kit Cat. Cat. #32028, Pierce, Rockford, Ill.) was applied for 30 minutes. To enhance this reaction, the cells were exposed to Avidin-Biotin complex for an additional 30 minutes (ImmunoPure ABC, peroxidase mouse IgG staining kit Cat. #32028, Pierce, Rockford, Ill.). The cells were then exposed to 20 mg of DAB (3,3-diaminobenzidine tetrahydrochloride No. D-5905, Sigma Chemical Co., St Louis, Mo., USA) and 0.033 ml of 3% H₂O₂ in 10 ml of saline for 10 minutes. The cells were washed in PBS, dehydrated in a series of ethanol, mounted and examined by light microscopy by an unbiased observer for the presence of a brown coloration indicating the presence of TNF-α.

Series-2, Neurophysiologic evaluation:

Thirteen pigs (body weight 25-30 kg) received an intramuscular injection of 20 mg/kg body weight of KETALAR® (ketamine, 50 mg/ml, Parke-Davis, Moms Plains, N.J.) and an intravenous injection of 4 mg/kg body weight of HYPNODIL® (methomidate chloride, 50 mg/ml, AB Leo, Helsingborg, Sweden) and 0.1 mg/kg body weight of STRESNIL® (azaperon, 2 mg/ml, Janssen Pharmaceutica, Beerse, Belgium). Anesthesia was maintained by additional intravenous injections of 2 mg/kg body weight of HYPNODIL® and 0.05 mg/kg body weight of STRESNIL®. The pigs also received an intravenous injection of 0.1 mg/kg of STESOLID NOVUM® (Diazepam, Dumex, Helsingborg, Sweden) after surgery.

Nucleus pulposus was harvested from the 5^{th} lumbar disc through a retro peritoneal approach (42). Approximately 40 mg of the nucleus pulposus was applied to the sacrococcygeal cauda equina through a midline incision and laminectomy of the first coccygeal vertebra. Four pigs did not receive any treatment (no treatment). Four other pigs received an intravenous infusion of 100 mg of doxycycline (Vibramycino, Pfizer Inc., New York, USA) in 100 mil of saline over 1 hour. In 5 pigs, the nucleus pulposus was mixed with 100 μl of a 1.11 mg/mL suspension of the anti-TNF-α antibody used in series 1, before application.

Three days after the application, the pigs were re-anesthetized by an intramuscular injection of 20 mg/kg body weight of KETALAR® and an intravenous injection of 35 mg/kg body weight 25 of PENTOTHAL® (Thiopental sodium, Abbott lab, Chicago, Ill.). The pigs were ventilated on a respirator. Anesthesia was maintained by an intravenous bolus injection of 100 mg/kg body weight of Chloralose ((a)-D(+)-gluco-chloralose, Merck, Damrstadt, Germany) and by a continuous supply of 30 mg/kg/hour of Chloralose. A laminectomy from the 4^{th} sacral to the 3^{rd} coccygeal vertebra was performed. The nerve roots were covered with SPONGOSTANE® (Ferrosan, Denmark). Local tissue temperature was continuously monitored and maintained at 37.5-38.0° C. by means of a heating lamp.

The cauda equina was stimulated by two E2 subdermal platinum needle electrodes (Grass Instrument Co., Quincy, Mass.) which were connected to a Grass SD9 stimulator (Grass Instrument Co., Quincy, Mass.) and gently placed intermittently on the cauda equina first 10 mm cranial and then 10 mm caudal to the exposed area. To ensure that only impulses from exposed nerve fibers were registered, the nerve root that exited from the spinal canal between the two stimulation sites were cut. An electromyogram (EMG) was registered by two subdermal platinum needle electrodes which were placed into the paraspinal muscles in the tail approximately 10 mm apart. This procedure is reproducible and represents a functional measurement of the motor nerve fibers of the cauda equina nerve roots. The EMG was visualized using a Macintosh IIci computer provided with Superscope software and MacAdios II AID converter (GW Instruments, Sommerville, Mass.) together with a Grass P18 preamplifier (Grass Instrument Co., Quincy, Mass.). The separation distance between the first peaks of the EMG from the two recordings was determined, and the separation distance between the two stimulation sites on the cauda equina was measured with calipers. The nerve conduction velocity between the two stimulation sites could thus be calculated from these two measurements.

The person performing the neurophysiologic analyses was unaware of the experimental protocol for the individual animal. After finishing the complete study, the data were arranged in the three experimental groups and statistical differences between the groups were assessed by Student's t-test. The experimental protocol for this experiment was approved by the local animal research ethics committee.

Series-3:

Thirteen pigs had autologous nucleus pulposus placed onto their sacrococcygeal cauda equina similar to series-2. Five pigs (bodyweight 25 kg) received the human/murine monoclonal antibody, REMICADE® (infliximab, Immunex Corporation, Seattle, Wash. 98101, USA) 100 mg i.v. preoperatively, and 8 pigs received ENBREL® (etanercept, Centocor B.V., Leiden, the Netherlands) 12.5 mg s.c. preoperatively and additionally 12.5 mg s.c. three days after the operation. Seven days after the nucleus pulposus-application the nerve root conduction velocity was determined over the application zone by local electrical stimulation according to series-2. To blind the study, the neurophysiological evaluation was conducted in parallel to another study and the person performing the analyses did not know from which study and what treatment each specific animal was subjected to. No non-treated animals were included in the series-3 due to the pre-existing knowledge of nerve conduction velocity after seven days of either nucleus pulposus or fat (control) application. The statistical difference between the groups, infliximab, and etanercept, nucleus pulposus without treatment (positive control from previous data) and application of retroperitoneal fat (negative control from previous data) was assessed by using ANOVA and Fisher's PLSD at 5%.

### Results

Series-1. Presence of TNF-α in pig nucleus pulposus-cells:

Examples of the light microscopic appearance of the stained glass slides. In the sections using BSA in PBS as “primary antibody” (control), no staining was observed, ensuring that there was no labeling and visualization of irrelevant antigens. When the anti-TNF-α antibody was applied at 1:40 dilution there was only weak staining. However, the staining increased with diminishing dilutions of the antibody. The staining was seen in the soma of the cells, and it was not possible to differentiate whether TNF-α was located in the cytoplasm, on the cell surface bound to the cell-membrane, or both.

Series-2. Neurophysiologic evaluation:

Application of non-modified nucleus pulposus and without any treatment induced a reduction in nerve conduction velocity similar to previous studies (Table 1). In contrast, treatment with doxycycline completely blocked this reduction (p<0.01 Student's t-test). Local application of anti-TNF-α-antibody also induced a partial block of this reduction, although not as complete as doxycycline and was not statistically significant as compared to the no treatment-series.

Series-3:

Treatment with both drugs seemed to prevent the nucleus pulposus-induced reduction of nerve root conduction velocities, since the average nerve conduction velocity for both these treatment groups were close to the average conduction of the fat-application series, as seen in a previous study (Table 2). The average nerve conduction velocity in pigs treated with ENBREL® was statistically different from the average nerve conduction velocity in the series with pigs with no treatment. The average new conduction velocity in the group treated with REMICADE® was also statistically significantly different from the average nerve conduction velocity in the group with no treatment.

TABLE 1
Series 2

| Treatment | n | NCV (m/s ± SD) |
|---|---|---|
| Local anti-TNF-α | 5 | 64 ± 28 |
| Doxycycline | 4 | 76 ± 9 |
| No treatment | 4 | 46 ± 12 |

TABLE 2
Series 3

| Treatment | n | NCV (m/s ± SD) |
|---|---|---|
| Fat* | 5 | 76 ± 11 |
| ENBREL ® | 8 | 78 ± 14 |
| REMICADE ® | 5 | 79 ± 15 |
| No treatment* | 5 | 45 ± 19 |

*Data included from ref. no. 42, Olmarker et al., 1993

### Discussion

The data of the present study demonstrated that TNF-α may be found in nucleus pulposus-cells of the pig. If TNF-α was blocked by a locally applied selective monoclonal antibody, the nucleus pulposus-induced reduction of nerve root conduction velocity was partially blocked, although not statistically significant as compared to the series with non-treated animals. However, if animals were treated systemically with doxycycline, infliximab, and etanercept to inhibit TNF-α, the reduction of nerve conduction velocity was significantly prevented.

In recent years, it has been verified that local application of autologous nucleus pulposus may injure the adjacent nerve roots. Thus, it has become evident that the nerve root injury seen as disc herniation may not be solely based on mechanical deformation of the nerve root, but may also be induced by unknown “biochemical effects” related to the epidural presence of herniated nucleus pulposus. Although this new research field has generated many experimental studies, the mechanisms and substances involved are not fully known. It has been seen that local application of autologous nucleus pulposus may induce axonal injury (24, 37, 38, 40-42), a characteristic injury of the myelin sheath (24, 38, 40-42), a local increase of vascular permeability (9, 36) infra vascular coagulations, reduction of infra neural blood flow (43), and leukotaxis (36). It has been seen that the nucleus pulposus-related effects may be blocked efficiently by methylprednisolone (38) and cyclosporin A (2), and slightly less efficiently by indomethacin (3), and lidocaine (69). Further, it has been understood that the effects are mediated by the nucleus pulposus-cells (37), particularly by substances or structures bound to the cell-membranes (25). When critically considering these data, it becomes evident that at least one specific cytokine could be related to these observed effects, Tumor Necrosis Factor-alpha (TNF-α). TNF-α may induce nerve injury (29, 31, 45, 50, 66), mainly seen as a characteristic myelin injury that closely resembles the nucleus pulposus-induced myelin-injury (29, 47, 51, 54, 62, 64, 66, 70). TNF-α may also induce an increase in vascular permeability (47, 66) and initiate coagulation (22, 34, 63). Further, TNF-α may be blocked by steroids (4, 8, 21, 61, 68), and cyclosporin A (11, 55, 67, 68). However, the blocking effect on TNF-α is not so pronounced by NSAID (14, 17, 20) and very low or the agonized by lidocaine (5, 32, 46, 60).

It was recently observed that local application of nucleus pulposus may induce pain-related behavior in rats, particularly thermal hyperalgesia (23, 40). TNF-α has also been found to be related to such pain-behavioristic changes (12, 35, 56,66), and also to neuropathies in general (30, 54, 56, 57). However there are no studies that have assessed the possible presence of TNF-α in the cells of the nucleus pulposus.

To assess if TNF-α could be related to the observed nucleus pulposus induced reduction in nerve root conduction velocity it was necessary first to analyze if there was TNF-α in the nucleus pulposus-cells. The data clearly demonstrated that TNF-α was present in these cells. TNF-α is produced as a precursor (pro-TNF) that is bound to the membrane, and it is activated by cleavage from the cell-membrane by a zinc-dependent metallo-endopeptidase (i.e., TNF-α converting enzyme, TALE) (6, 15, 16, 48, 49). This may thus relate well to experimental findings, where application of only the cell-membranes of autologous nucleus pulposus-cells induced nerve conduction velocity reduction, which indicated that the effects were mediated by a membrane-bound substance. Second, the effects of the TNF-α had to be blocked in a controlled manner. We then first chose to add the same selective antibody that was used for immunohistochemistry in series 1, which is known to also block the effects of TNF-α, to the nucleus pulposus before application. Also, we chose to treat the pigs with doxycycline, which is known to block TNF-α (26, 27, 33, 52, 53). However, due to the low pH of the doxycycline preparation, it was chosen to treat the pigs by intravenous injection instead of local addition to the nucleus pulposus since nucleus pulposus at a low pH has been found to potentiate the effects of the nucleus pulposus (38, 39).

Two recently developed drugs for specific TNF-α inhibition were also included in the study. Infliximab is a chimeric monoclonal antibody composed of human constant and murine variable regions. Infliximab binds specifically to human TNF-α. As opposed to the monoclonal antibody used in series-2 for the 3-day observation period, infliximab was not administered locally in the autotransplanted nucleus pulposus, but instead was administered systemically in a clinically recommended dose (4 mg/kg).

Etanercept is a dimeric fusion protein consisting of the Fc portion of human IgG. The drug, etanercept, was administered in a dosage comparable to the recommended dose for pediatric use (0.5 mg/kg, twice a week).

The data regarding nerve conduction velocity showed that the reduction was completely blocked by the systemic-treatment and that the nerve conduction velocities in these series were close to the conduction velocity after application of a control substance (retro peritoneal fat) from a previous study (42). Application of the anti-TNF-α-antibody to the nucleus pulposus also partially prevented the reduction in nerve conduction velocity. However, the reduction was not as pronounced as that observed for doxycycline, and the velocity in this series was not statistically different to the velocity in the series with untreated animals, given the wide deviation of the data.

The local anti-TNF-α antibody treatment only partially blocked the nucleus pulposus-induced reduction of nerve conduction velocity and the high standard deviation of the data could probably have at least three different explanations. First, if looking at the specific data within this group, it was found that the nerve conduction velocity was low in 2 animals (mean 37.5 m/s) and high in 3 animals (mean 81.3 m/s). There are thus 2 groups of distinctly different data within the anti-TNF-α treatment series. This will account for the high standard deviation and might imply that the blocking effect was sufficient in 3 animals and insufficient in 2 animals. The lack of effects in these animals could be based simply on the amount of antibodies in relation to TNF-α molecules not being sufficient, and if a higher dose of the antibody had been used, the TNF-α effects would thus have been blocked even in these animals. Such a scenario could then theoretically imply that TNF-α alone is responsible for the observed nucleus pulposus-induced effects, and that this could not be verified experimentally due to the amount of antibody being too low.

Second, it is also known that tetracyclines such as doxycycline and minocycline may block a number of cytokines and other substances. For instance they may block IL-1 (1, 28, 58), IFNg (27), NO-synthetases, and metalloproteinases (1, 53, 58). Particularly IL-1 and IFN-γ are known to act synergistically with TNF-α and are known to be more or less neurotoxic (7, 10, 13, 18, 19, 56, 59). These substances are also blocked by steroids and cyclosporin A, which corresponds with the previous observations on nucleus pulposus-induced nerve root injury that have shown that the nucleus pulposus-induced effects may be blocked by these substances (8, 67). One may therefore also consider the possibility that a selective block of TNF-α may not be sufficient to completely block the nucleus pulposus-induced effects on nerve function, and that the simultaneous block of other synergistic substances is necessary as well. Thus, this scenario, on the other hand, implies that TNF-α may not solely be responsible for the nucleus pulposus-induced effects, and that other synergistic substances, which are also blocked by doxycycline, also may be necessary.

The third explanation could be that the amount of TNF in the nucleus pulposus may well be enough to start the pathophysiologic cascade locally in the nerve root. The cascade comprises increased vascular permeability and aggregation and recruitment of systemic leukocytes. However, it is these leukocytes which contain the major concentration of TNF-α and that systemic treatment in a sufficient dose is necessary to block the contribution from these leukocytes, and thereby also blocking the events leading to nerve injury.

TNF-α may have various pathophysiologic effects. It may have direct effects on tissues such as nerve tissue and blood vessels, it may trigger other cells to produce other pathogenic substances and it may trigger release of more TNF-α both by inflammatory cells and also by Schwann-cells locally in the nerve tissue (65). There is thus reason to believe that even low amounts of TNF-α may be sufficient to initiate these processes and that there is a local recruitment of cytokine producing cells and a subsequent increase in production and release of other cytokines as well as TNF-α. TNF-α may therefore act as the “ignition key” of the pathophysiologic processes and play an important role for the initiation of the pathophysiologic cascade behind the nucleus pulposus-induced nerve injury. However, the major contribution of TNF-α may be derived from recruited, aggregated and maybe even extravasated leukocytes, and that successful pharmacologic block may be achieved only by systemic treatment.

In conclusion, for the first time a specific substance (TNF-α) has been linked to the nucleus pulposus-induced nerve root injury. This new information may be of significant importance for the continued understanding of nucleus pulposus-induced nerve injury as well as raising the question of the potential future clinical use of pharmacological interference with TNF-α and related substances, for treatment of sciatica.

The presence of TNF-α in pig nucleus pulposus-cells was thus immunohistochemically verified. Block of TNF-α by a locally applied monoclonal antibody partially limited the nucleus pulposus-induced reduction of nerve root conduction velocity, whereas intravenous treatment with doxycycline, infliximab, and etanercept significantly blocked this reduction. These data for the first time links one specific substance, TNF-α, to the nucleus pulposus-induced nerve injury.

Aminoguanidine has showed to inhibit the release of nitrogen oxide (NO) at nerve root injuries by inhibiting inducible nitrogen oxide synthetase, and aminoguanidine is thus one compound that inhibits a compound triggered by the release of TNF-α.

### Example 3

### CDP-571 (HUMICADE®)

For an individual presenting with, for example, radiating pain corresponding to the left 4th lumbar nerve root due to sciatica, the following treatment can be used. The person can be treated with 10 mg/kg of CDP-571 (HUMICADE®) intravenously in a single dose. The person can then be monitored to determine whether additional drugs need to be administered.

### Example 4

### D2E7

For an individual, for example a woman, presenting with radiating pain and slight nerve dysfunction corresponding to the 1st sacral nerve on the left side due to disc herniation with sciatica, the following treatment plan can be used. The individual can be administered D2E7 intravenously at a dosage of 5 mg/kg body weight. The woman's condition could then be followed, for example, at 4 to 8 week intervals after her first injection. Further D2E7 administration would be determined based on the clinical needs of the patient, as determined by the clinician.

### Example 5

### CDP-870

For another individual, who for example, presented with dermatomal pain corresponding to the first sacral nerve root on the left side, the following treatment regimen can be used. The individual can be administered intravenously an injection of 5 mg/kg body weight of CDP-870. The progress of the patient is then followed and additional injections determined by the clinician based on clinical presentation of the patient.

### References

1. Amin A R, Attur M G, Thakker G D, Patel P D, Vyas P R, Patel R N, Patel I R, Abramson S B, A novel mechanism of action of tetracyclines: effects on nitric oxide syntheses, Proc Natl Acad Sci U S A 1996; 93: 14014-9.

2. Arai I, Konno S, Otani K, Kikuchi S, Olmarker K, Cyclosporin A blocks the toxic effects of nucleus pulposus on spinal nerve roots, Submitted.

3. Arai I, Mao G P, Otani K, Komo S, Kikuchi S, Olmarker K, Indomethacin blocks nucleus pulposus related effects in adjacent nerve roots, Accepted for publication in Eura Spine J.

4. Baumgartner R A, Deramo V A, Beaven M A, Constitutive and inducible mechanisms for synthesis and release of cytokines in immune cell lines, J Immunol 1996; 157: 4087-93.

5. Bidani A, Herning T A, Effects of lidocaine on cytosolic pH regulation and stimulus-induced effector functions in alveolar macrophages, Lung 1997; 175: 349-61.

6. Black R A, Rauch C T, Kozlosky C J, Peschon I J, Slack J L, Wolfson M F, Castner B J, Stocking K L, Reddy P, Srinivasan S, Nelson N, Boiani N, Schooley K A, Gerhart M, Davis R, Fitzner J N, Johnson R S, Paxton R J, March C J, Cerretti D P, A metalloproteinase disintegrin that releases tumour-necrosis factor- αfrom cells, Nature 1997; 385: 729-33.

7. Bluthe R M, Dantzer R, Kelley K W, Interleukin-I mediates behavioural but not metabolic effects of tumor necrosis factor alpha in mice, Eur J Pharmacol 1991; 209: 281-3.

8. Brattsand R, Linden M, Cytokine modulation by glucocorticoids: mechanisms and actions in cellular studies, Aliment Pharmacol Ther 1996; 10: 81-90.

9. Byröd G, Otani K, Brisby H, Rydevik B, Olmarker K, Methylprednisolone reduces the early vascular permeability increase in spinal nerve roots induced by epidural nucleus pulposus application, J Orthop Res 1987; 18: 6: 983-7.

10. Chao C C, Hu S, Ehrlich L, Peterson P K, Interleukin-1 and tumor necrosis factor-alpha synergistically mediate neurotoxicity: involvement of nitric oxide and of N-methyl-D-aspartate receptors, Brain Behav Immun 1995; 9: 355-65.

11. Dawson J, Hurtenbach U, MacKenzie A, Cyclosporin A inhibits the in vivo production of interleukin-Ibeta and tumour necrosis factor alpha, but not interleukin-6, by a T-cell independent mechanism, Cytokine 1996; 8: 882-8.

12. DeLeo J A, Colbum R W, Rickman A J, Cytokine and growth factor immunohistochemical spinal profiles in two animal models of mononeuropathy, Brain Res 1997; 759: 50-7.

13. Gadient R A, Cron K C, Otten U, Interleukin-I beta and tumor necrosis factor-alpha synergistically stimulate nerve growth factor (NGF) release from cultured rat astrocytes, Neurosci Lett 1990; 117: 335-40.

14. Garcia-Vicuna R, Diaz-Gonzalez F, Gonzalez-Alvaro 1, del Pozo Na, Moilinedo F, Cabanas C, Gonzalez-Amaro R, Sanchez-Madrid F, Prevention of cytokine-induced changes in leucocyte adhesion receptors by nonsteroidal antiinflammatory drugs from the oxicam family, Arthritis Rheum 1997; 40: 143-53.

15. Gearing A J, Beckett P, Christodoulou M, Churchill M, Clements J, Davidson A H, Drummond A H, Galloway W A, Gilbert R, Gordon J L, et al., Processing of tumour necrosis factor-alpha precursor by metalloproteinases, Nature 1994; 370: 555-7.

16. Gazelle E J, Banda M J, Leppert D, Matrix metallo-proteinases in immunity, J Immunol 1996; 156: 14.

17. Gonzalez E, de la Cruz C, de Nicolas R, Egido J, Herrero-Beaumont G, Long-term effect of nonsteroidal anti-inflammatory drugs on the production of cytokines and other inflammatory mediators by blood cells of patients with osteosis, Agents Actions 1994; 41: 171-8.

18. Hartung B P, Jung S, Stoll G, Zielasek J, Schmidt B, Archelos J J, Toyka K V, Inflammatory mediators in demyelinating disorders of the CNS and PNS, J Neuroinununol 1992; 40: 197-210.

19. Hattori A, Iwasald S, Murase K, Tsujimoto M, Sato M, Hayashi K, Kohno M, Tumor necrosis factor is markedly synergistic with interleukin I and ii3terferon-gamma in stimulating the production of nerve growth factor in fibroblasts, FEBS Lett 1994; 340: 177-80.

20. Herman J H, Sowder W G, Hess E V, Nonsteroidal antuinflammatory drug modulation of prosthesis pseudomembrane induced bone resorption, J Rheunutol 1994; 21: 338-43.

21. Iwamoto S, Takeda K, Possible cytotoxic mechanisms of TNF in vitro, Hum Cell 1990; 3: 107-12.

22. Jurd K M, Stephens C J, Black M M, Hunt B J, Endothelial cell activation in cutaneous vasculitis, Clin Exp Dermatol 1996; 21: 28-32.

23. Kawakami M, Tamaki T, Weinstein J N, Hashizume H, Nishi H, Meller S T, Pathomechanism of pain-related behaviour produced by allografts of intervertebral disc in the rat, Spine 1996; 21: 2101-7.

24. Kayama S, Konno S, Olmarker K, Yabuki S, Kikuchi S, Incision of the anulus fibrosis induces nerve root morphologic, vascular, and functional changes. An experimental study, Spine 1996; 21: 2539-43.

25. Kayama S, Olmarker K, Larsson K, Sjören-Jansson E, Lindahl A, Rydevik D, Cultured, autologous nucleus pulposus cells induce structural and functional changes in spinal nerve roots, Spine, 1998; 23:90: 2155-58,

26. Kloppenburg M, B-an B M, de Rooij-Dijk H H, Mitenburg A M, Daha M R, Breedveld F C, Dijkmans B A, Verweij C, The tetracycline derivative minocycline differentially affects cytokine production by monocytes and T lymphocytes, Antimicrob Agents Chemother 1996; 40: 934-40.

27. Kloppenburg M, Verweij C L, Miltenburg A M, Verboeven A J, Daha M R, Dijlanans B A, Breeveld F C, The influence of tetracyclines on T cell activation, Clin EU Immunol 1995; 102: 635-41.

28. Lamster M, Pullman J R, Celenti R S, Grbic J T, The effect of tetracycline fiber therapy on beta-glucuronidase and interleukin-I beta in crevicularfluid, J Clin Periodontol 1996; 23: 816-22.

29. Liberski P P, Yanagihara R, Nerurkar V, Gajdusek D C, Further ultrastructural studies of lesions induced in the optic nerve by tumor necrosis factor alpha (TNF-m): a comparison with experimental Creutzfeldt-Jakob disease, Acta Neurobiol En (Warsz) 1994; 54: 209-18.

30. Lin X H, Kashiina Y, Khan M, Heller K B, Gu X Z, Sadun A A, An immunohistochemical study of TNF-α in optic nerves from AIDS patients, Curr Eye Res 1997; 16: 1064-8.

31. Madigan M C, Sadun A A, Rao N S, Dugel P U, Tenhula W N, Gill P S, Tumor necrosis factor-alpha (TNF-α)-induced optic neuropathy in rabbits, Neurol Res 1996; 18: 176-84.

32. Matsumori A, Ono K, Nishio R, Nose Y, Sasayaina S, Amiodarone inhibits production of tumor necrosis factor-alpha by human mononuclear cells: a possible mechanism for its effect in heart failure, Circulation 1997; 96: 1386-9.

33. Milano S, Arcoleo F, D'Agostino P, Cillari E, Intraperitoneal injection of tetracyclines protects mice from lethal endotoxemia downregulating inducible nitric oxide synthase in various organs and cytokine and nitrate secretion in blood, Antimicrob Agents Chemother 1997; 41: 117-21.

34. Nawroth P, Handley D, Matsueda G, De Waal R, Gerlach H, Blohm D, Stem D, Tumor necrosis factor/cachectin-induced intra vascularfibrin formation in meth A fibrosarcomas, J Exp Med 1988; 168: 637-47.

35. Oka T, Wakugawa Y, Hosoi M, Oka K, Hari T, Intracerebroventricular injection of tumor necrosis factor-alpha induces thermal hyperalgesia in rats, Neuroimmunomodulation 1996; 3: 135-40.

36. Olmarker K, Blomquist J, Stromberg J, Nanmnark, U, Thomsen P, Rydevik B, Inflamma-togenic properties of nucleus pulposus, Spine 1995; 20: 665-9.

37. Olmarker K, Brisby H, Yabuki S, Nordborg C, Rydevik B, The effects of normal, frozen, and hyaluronidase-digested nucleus pulposus on nerve root structure and function, Spine 1997; 22: 4715; discussion 476.

38. Olmarker K, Byrod G, Comefjord M, Nordborg C, Rydevik B, Effects of methylprednisolone on nucleus pulposus-induced nerve root injury, Spine 1994; 19: 1803-8.

39. Iwabushi M, Rydevik B, Kikuchi S, Olmarker K, Methylprednisolone reduces the early vascular permeability increase in spinal nerves by epidural nucleus pulposus application, Accepted for publication in Spine.

40. Olmarker K, Myers R R, Pathogenesis of sciatic pain: Role of herniated nucleus pulposus and deformation of spinal nerve root and DRG, Pain, 1998, 78: 9-105.

41. Olmarker K, Nordborg C, Larsson K, Rydevik B, Ultrastrnctural changes in spinal nerve roots induced by autologous nucleus pulposus, pine 1996; 21: 411-4.

42. Olmarker K, Rydevik B, Nordborg C, Autologous nucleus pulposus induces neurophysiologic and histologic changes in porcine cauda equina nerve roots, Spine 1993; 18: 1425-32.

43. Otani K, Arai I, Mao G P, Konno S, Ohnarker K, Kikuchi S, Nucleus pulposus-induced nerve root injury. The relationship between blood flow and nerve conduction velocity, Neurosurgery 1999; 45: 619-20

45. Petrovich M S, Hsu H Y, Gu X, Dugal P, Heller K B, Sadun A A, Pentoxifylline suppression of TNF- alpha mediated axonal degeneration in the rabbit optic nerve, Neurol Res 1997; 19: 551-4.

46. Pichler W J, Zanni M, von Greyerz S, Schnyder B, Mauri-HeUweg D, Wendland, T, High IL-5 production by human drug-specific T cell clones, Int Arch Allerity Immunol 1997; 1 13: 177-80.

47. Redford E J, Hall S M, Smith K J, Vascular chances and demyelination induced by the intra neural injection of tumour necrosis factor, Brain 1995; 1 18: 869-78.

48. Robache-Gallea S, Bruneau J M, Robbe H, Morand V, Capdevila C, Bhatnagar N, Chouaib S, Roman-Roman S, Partial purification and characterization of a tumor necrosis factor- alpha converting activity, Eur J Immunol 1997; 27: 1275-82.

49. Rosendahl M S, Ko S C, Long D L, Brewer M T, Rosenzweig D, Hedl E, Anderson L, Pyle S M, Moreland J, Meyers M A, Kohno T, Lyons D, Lichenstein H S, Identification and characterization of a pro-tumor necrosis factor- alpha-processing enzyme from the ADAM family of zinc metalloproteases, J Biol Chem 1997; 272: 24588-93.

50. Said G, Hontebeyrie-Joskowicz M, Nerve lesions induced by macrophage activation, Res Immunol 1992; 143: 589-99.

51. Sehnaj K W, Raine C S, Tumor necrosis factor mediates myelin and oligodendrocytc damage in vitro, Ann Neurol 1988; 23: 339-46.

52. Shapira L, Houri Y, Barak V, Halabi A, Soskoine W A, Stabholz A, Humnan monocyte response to cementum extracts from periodontally diseased teeth: effect of conditioning with tetracycline, J Periodontol 1996; 67: 682-7.

53. Shapira L, Houri Y, Barak V, Soskolne W A, Halabi A, Stabholz A, Tetracycline inhibits' Porphyromonas gingivalis lipopolysaccharide- induced lesions in vivo and TNF α processing in vitro, J Periodontal Res 1997; 32: 183-8.

54. Sharief M K, Ingram D A, Swash M, Circulating tumor necrosis factor-alpha correlates with electrodiagnostic abnormalities in Guillain-Barre syndrome, Ann Neurol 1997; 42: 6873.

55. Smith C S, Ortega G, Parker L, Shearer W T, Cyclosporin A blocks induction of tumor necrosis factor-alpha in human B lymphocytes, Biochem Bionhys Res Commun 1994; 204: 383-90.

56. Sommer C, Schmidt C, George A, Toyka K V, A metalloprotease-inhibitor reduces pain associated behaviour in mice with experimental neuropathy, Neurosci Lett 1997;237:45-8.

57. Sorkin L S, Xiao W H, Wagner R, Myers R R, Tumour necrosis factor-alpha induces ectopic activity in nociceptive primary afferentfibres, Neuroscience 1997; 81: 255-62.

58. Steinrneyer J, Daufeldt S, Taiwo Y O, Pharmacological effect of tetracyclines on proteoglycanases from interleukin-l-treated articular cartilage, Biochem Pharmacol 1998; 55: 93-100.

59. Stoll G, Jung S, Jander S, van der Meide P, Hartung B P, Tumor necrosis factor-alpha in immunomediated demyelination and Wallerian degeneration of the rat peripheral nervous system, Neuroimmunol 1993; 45: 175-82.

60. Takao Y, Mikawa K, Nishina Y, Maekawa N, Obara H, Lidocaine attenuates hyperoxic lung injury in rabbits, Acta Anaesthesiol Scand 1996; 40: 318-25.

61. Teoh K H, Bradley C A, Galt J, Burrows H, Steroid inhibition of cytokine-mediated vasodilation after warm heart surgery, Circulation 1995; 92: 11347-53.

62. Tsukmnoto T, Ishikawa M, Yamamoto T, Suppressive effects of TNF-α on myelinformation in vitro, Acta Neurol Scand 1995; 91: 71-5.

63. van der Poll T, Jansen P M, Van Zee K J, Welborn M Br, de Jong I, Hack C E, Loetscher H, Lesslauer W, Lowry S F, Moidawer L L, Tumor necrosis factor-alpha induces activation of coagulation and fibrinolysis in baboons through an exclusive effect on the p55 receptor, Blood 1996; 88: 922-7.

64. Villarroya H, Violleau K, Ben Younes-Chennoufi A, Baumann N, Myelin-induced experimental allergic encephalomyelitis in Lewis rats: tumor necrosis factor alpha levels in serum of cerebrospinal fluid immunohistochemical expression in glial cells and neurophages of optic nerve and spinal cord, J Neuroimmunol 1996; 64: 55-61.

65. Wagner R, Myers R R, Schwann cells produce tumor necrosis factor alpha: expression in injured non-injured nerves, Neuroscience 1996; 73: 625-9.

66. Wagner R, Myers R R, Endoneurial injection of TNF-α produces neuropathic pain behaviours, Neuroreport 1996; 7: 2897-901.

67. Wasaki S, Sakaida I, Uchida K, Kiinura T, Kayano K, Oldta K, Preventive effect of cyclosporin A on experimentally induced acute liver injury in rats, Liver 1997; 17: 107-14.

68. Wershil B K, Furuta G T, Lavigne J A, Choudhury A R, Wang Z S, Galli S J, Dexamethasone cyclosporin A suppress mast cell-leukocyte cytokine cascades by multiple mechanisms, Int Arch Allergy Immunol 1995; 107: 323-4.

69. Yabuki S, Kawaguchi Y, Olmarker K, Rydevik B, Effects of lidocaine on nucleus pulposus-induced nerve root injury, Spine, 1998; 23: 29: 2383-89

70. Zhu J, Bai X F, Mix E, Link H, Cytokine dichotomy in peripheral nervous system influences the outcome of experimental allergic neuritis: dynamics of MRNA expression for IL-lbeta, IL-6, IL-10, IL-12, TNF-α, TNF-beta, and cytolysin, Clin Immunol Immunopathol 1997; 84: 85-94.

1. A method for inhibiting the action of TNF-α for treating nerve disorders in a subject by administering a TNF-α inhibitor comprising administering to said subject a therapeutically effective dosage of said TNF-α inhibitor wherein said TNF-α inhibitor is CDP-571 (HUMICADE™), D2E7, or CDP-870.

2. The method of claim 1, wherein the subject is a vertebrate.

3. The method of claim 2, wherein the vertebrate is a mammal.

4. The method of claim 3, wherein the mammal is a human.

5. The method of claim 1, wherein said nerve disorder is a spinal disorder.

6. The method of claim 1, wherein said nerve disorder is nerve root injury.

7. The method of claim 1, wherein said nerve disorder is caused by herniated discs.

8. The method of claim 1, wherein said nerve disorder is sciatica.

9. The method of claim 1, wherein said nerve disorder involves pain.

10. The method of claim 1, wherein said nerve disorder is nucleus pulposus-induced nerve injury.

11. The method of claim 1, wherein said nerve disorder is spinal cord compression.

12. The method of claim 1, wherein said TNF-α inhibitor is administered systemically or locally.

13. The method of claim 1, wherein said TNF-α inhibitor is administered parenterally.

14. The method of claim 1, wherein said TNF-α inhibitor is administered intramuscularly, intravenously, subcutaneously, orally, or rectally.

15. The method of claim 14, wherein said TNF-α inhibitor is administered intravenously by injection or infuision.

16. The method of claim 15, wherein said TNF-α inhibitor is administered orally at a dosage of about 20 mg to about 1,500 mg.

17. The method of claim 1, wherein the TNF-α is D2E7 and is administered in a dosage of about 0.1 mg/kg to about 50 mg/kg body weight of said subject.

18. The method of claim 1, wherein the TNF-α is CDP-870 and is administered in a dosage of about 1 mg/kg to about 50 mg/kg body weight of said subject.

19. A method for inhibiting the action of TNF-α for treating nerve disorders in a subject by administering a TNF-α inhibitor comprising administering to said subject a therapeutically effective dosage of said TNF-α inhibitor wherein said TNF-α inhibitor is a lactoferrin, CT3, ITF-2357, PD-168787, CLX-1100, M-PGA, NCS-700; PMS-601, RDP-58, TNF-484A, PCM-4, CBP-1011, SR-31747, AGT-1, Solimastat, CH-3697, NR58-3.14.3, RIP-3, Sch-23863, or SH-636.

20. A pharmaceutical composition for treating nerve disorders in a subject comprising a therapeutically effective amount of a TNF-α inhibitor wherein said TNF-α inhibitor is CDP-571 (HUMICADE™), D2E7, or CDP-870, and a pharmaceutically acceptable carrier, and wherein said pharmaceutical composition inhibits nerve injury when administered to said subject.

21. The pharmaceutical composition of claim 20, wherein the subject is a vertebrate.

22. The pharmaceutical composition of claim 21, wherein the vertebrate is a mammal.

23. The pharmaceutical composition of claim 20, wherein the mammal is a human.

24. The pharmaceutical composition of claim 20, wherein said monoclonal antibody is D2E7 in a dosage amount of about 0.1 mg/kg to about 50 mg/kg body weight of said subject.

25. The pharmaceutical composition of claim 20, wherein said monoclonal antibody CDP-870 in an amount of about 1.0 mg/kg to about 50 mg/kg body weight of said subject.

26. The pharmaceutical composition of claim 20, wherein said nerve disorder is selected from the group consisting of a spinal disorder, a nerve root injury, a nerve disorder caused by herniated discs, a nerve disorder involving pain, a nucleus pulposus-induced nerve injury, a spinal cord compression, and sciatica.

27. The pharmaceutical composition of claim 20, wherein said pharmaceutical composition is formulated for intravenous, intramuscular, oral, rectal, or subcutaneous administration.

28. The pharmaceutical composition of claim 20, wherein said pharmaceutical composition is formulated for parenteral administration.

29. A pharmaceutical composition for treating nerve disorders in a subject comprising a therapeutically effective amount of a TNF-α inhibitor wherein said TNF-α inhibitor is a lactoferrin, CT3, ITF-2357, PD-168787, CLX-1100, M-PGA, NCS-700; PMS-601, RDP-58, TNF-484A, PCM-4, CBP-1011, SR-31747, AGT-1, Solimastat, CH-3697, NR58-3.14.3, RIP-3, Sch-23863, or SH-636, and a pharmaceutically acceptable carrier, and wherein said pharmaceutical composition inhibits nerve injury when administered to said subject.
